(19) United States
(12) Patent Application Publication
(10) Pub. No.: US 2007/0191675 A1
(43) Pub. Date: Aug. 16, 2007

(54) Actinic light colposcope and method to detect lesions in the lower female genital tract produced by human papilloma virus using an actinic light colposcope

(76) Inventor: Joel Gerardo Diaz Sanchez, Mexico City (MX)

Correspondence Address: Alberto A. Leon, Esq.;BAUMAN, DOW & LEON, P.C., P.O. Box 30684, Albuquerque, NM, 87190, US

(21) Appl. No.: 11/352,540

(22) Filed: Feb. 13, 2006

(51) Int. Cl. A61B 1/00

(52) U.S. Cl. 600/105

(57) ABSTRACT

The present invention involves an apparatus and method to detect lesions in the lower female genital tract produced by the Human Papilloma Virus using an actinic light colposcope. The apparatus of the present invention comprises mechanical, electrical and optical (also referred to herein as the actinic light) components. The method of the present invention comprises the general steps of: (1) illumination, (2) excitement, and (3) suppression. The method disclosed and claimed herein in the clinical context takes between 15 and 20 minutes and reliable results are obtained during the patient's office visit.

### RELATED APPLICATIONS

Applicant claims the benefit of PCT Application No. PCT/MX 03/0088 filed with the PCT Receiving Office in Mexico City, Mexico on Oct. 27, 2003.

### ENTERING THE NATIONAL STAGE IN THE U.S.

This application constitutes the entry of the National Stage in the U.S. of Application No. PCT/MX 03/0088 pursuant to 35 U.S.C. §371. Applicant is attaching hereto copy of PCT Application No. PCT/MX 03/0088 pursuant to C.F.R. §1.490(b) (1).

### TECHNICAL FIELD OF THE INVENTION

Gynecological tools and methods, gynecological detection methods, tools and methods to detect lower female genital tract lesions, actinic light-based tools and methods to detect lower female genital tract lesions.

### BACKGROUND OF THE INVENTION

The viral gender Papilloma is an extremely extensive DNA virus, which infects many animal species populations, including humans. There are over 100 types of known Papilloma viral sources, which when infecting humans are known as Human Papilloma Virus (HPV). The interest in those viruses has been gradually increasing since 1970, when for the first time a function was attributed in the etiology of the Uterine Cervix Cancer.

Cervical Uterine Cancer is a common type of cancer in women, which results from a change of the epithelial cell tissue of the cervix, vaginal walls and vulva. Those cells are normal initially but gradually turn pre-cancerous. Before cancerous cells are found in the cervix, the cervical tissue undergoes changes and abnormal cells start to appear. The process is known as Dysplasia or Cervical Intraepithelial Neoplasia (“CIN”).

More than 30 types of viral HPV exist which can infect the female lower genital tract. There are benign types of these viruses that are called “low risk,” and other oncogenics called “high risk.”

Uterine Cervix Cancer is one of the most common forms of cancers among women. It is estimated that there are over 500,000 cases of Uterine Cervix Cancer per year worldwide. Nearly 80% of these cases are reported in developing countries. The high incidence of Uterine Cervical Cancer reflects the deficiencies of early detection programs.

The high-risk type of virus in the American Continent has been the HPV 16; identified as the Asian-American variety in about 50% of the reported cases.

Starting in 1990 there has been an increase in research geared toward early detection of Uterine Cervical Cancer. New detection methods are being tested such as fluorescence spectroscopy, cytological techniques, and various testing methods using molecular biology.

The tool used for the observation and diagnosis of many female lower genital tract diseases is called a colposcope. The examination/detection technique using a colposcope is known as colposcopy. More specifically, colposcopy is the procedure used in gynecology to examine the epithelium of the female inferior genital tract using the accumulated knowledge in the field to evaluate its illnesses. A colposcope is similar to a stereoscopic microscope, to the extent that it is an optical system that modulates the photogenic energy through which it enlarges an image. The colposcope was invented in 1925 in Germany.

The colposcopy technique generally comprises the steps of: (1) inserting a vaginal mirror while having the patient in the gynecological position; (2) placing the colposcope a few centimeters off the vaginal introitus in such a way that the tool does not touch the patient; and (3) observing the desired tissue, its morphology and its vascular patterns.

HPV induced lesions and CINs are not often easily diagnosed using the procedure outlined above. The main reason is that the common clinical environment usually lacks the equipment and therefore the tests to facilitate the easy diagnosis of those conditions. Accordingly, many gynecologists have to use laboratory tests with wide margins of error, or use highly sophisticated and costly equipment, e.g., spectroscopy. Such equipment usually requires a large investment of time and resources to train personnel, long waiting periods to obtain results and multiple visits by the patient.

The present invention presents a radical advance in the field, as well as a practical solution to the shortcomings of the prior art in the fields of instrumentation and methods of diagnosis of lesions caused by HPV. As set forth above, the HPV is the etiological agent of most neoplasias and Uterine Cervical Cancers. Uterine Cervical Cancer is the number one cause of death through cancer among women between the third and sixth decade of life. That disease is also the second most frequent malignant neoplasia in the world, with an incidence in the industrialized countries of 10 per 100,000 inhabitants. In developing countries the incidence quadruples to 40 per 100,000 inhabitants. More specifically, in Mexico among women older than 25 years of age, the incidence of Uterine Cervical Cancer is 50 per 100,000 inhabitants and 16,000 new cases are detected each year according to the statistics of the Mexican Health Department. Every 2 hours a Mexican woman dies of Cervical Cancer. In short, the HPV is the most important risk factor for CIN.

The apparatus and method of the present invention avoids costly medical tests and provides quick and highly reliable diagnostic results for the detection of HPV in the female lower genital tract. In fact, the present invention allows excellent and reliable results to be obtained at the same time the tests are being conducted and in the same location.

There are additional, distinct advantages presented by the apparatus and method of the present invention, among them: (1) portability; (2) ergonomical design for the user and patient; (3) ease of use; (4) low heat intensity light source; (5) works dually like a common colposcope and like an actinic light colposcope; and (6) is specific to detect lesions caused by HPV by virtue of its spectral band with a long-lasting light with a specific wave for the fluorochrome isothiocyanate of fluoresceine (“FITC”); (7) quick results during the same visit and at the same location where the test is performed; and (8) significantly lower costs and less taxing testing requirements for user and patient alike.

### BRIEF DESCRIPTION OF DRAWINGS

FIG. 1 is a schematic illustrating optical elements of the invention.

FIG. 2 is a side view of a housing for the optical components of FIG. 1.

FIG. 3 is a schematic illustrating a first collector lens.

FIG. 4 is a schematic illustrating optical components and a focal point at the cervix.

FIG. 5 is an illustration of an optical wave.

FIG. 6 is an illustration of optical wavelength.

FIG. 7 is an illustration of optical wavelength.

FIG. 8 is a representation of incident light and fluorescent emission in a cell.

FIG. 9 is a representation of atom showing excitement of electrons.

FIG. 10 is a representation of the detection of fluorescent emission in a cell.

FIG. 11 is a representation of fluorescent detection in a cell.

### SUMMARY OF THE INVENTION

The explanations herein below, along with the accompanying figures, clearly describe in detail the characteristics of the new and useful apparatus and method of the present invention.

The apparatus of the present invention comprises mechanical, electrical and optical (also referred to herein as the actinic light) components.

The mechanical component further comprises a base or support, an arm, a bolster. The base or support comprises a rectangular steel plate, which provides overall stability to the equipment; four independent rotating wheels further comprising a brake mechanism which allows the equipment to be moved and set in place; a horizontal support further comprising a rotating articulation to maneuver and set the optical handle and bolster, and further holding in place the feeding source for the illumination equipment.

The arm comprises a counterbalanced mechanism which allows tri-axial movement, which in turn allows the user of the present invention to easily set the equipment in the precise location required by the gynecological procedure being conducted.

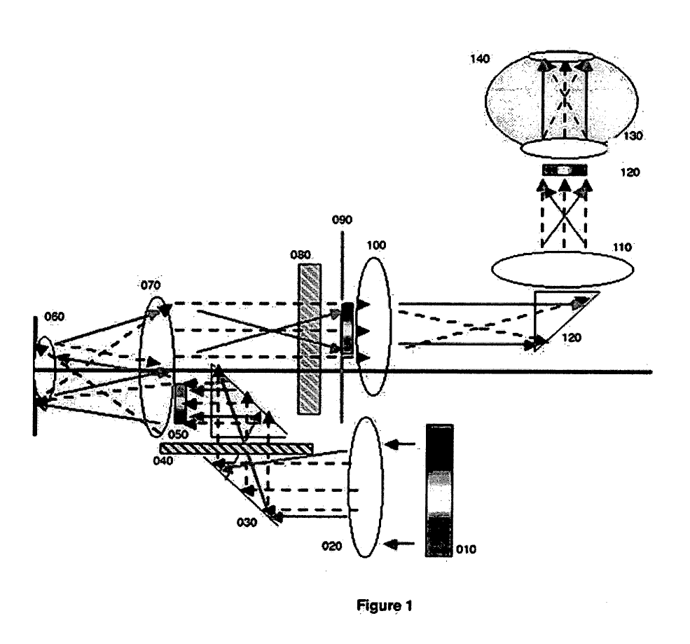

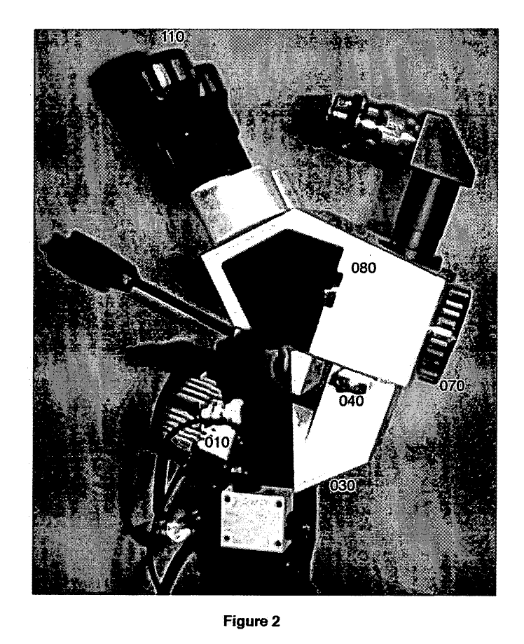

The bolster is the central and, in some respects, the most functionally important element of the apparatus, as illustrated in FIGS. 1 and 2. The bolster contains most of the elements that constitute the “new” colposcope. The bolster generally comprises: (1) a focusing system which further comprises a mechanism which allows finely calibrated movement; and (2) an optical source of actinic light which further comprises an exciting filter (FIG. 1, 2,7 040), and suppressing filters (FIG. 1,2,10 080). The bolster is a fundamental component of the apparatus of this invention because therein takes place the transmission and modulation of luminescent energy, which allows the user to see cervical images which can then be photographed

The electronic component of the present invention comprises: (1) a power source that feeds a bulb (halogen photo optic lamp Xenophot HLX 64610 Osram 12 volts 50 watts); (2) two connectors of 120 V, alternate current suitable for a video camera and recorder; (3) and a contact of 12 V, direct current for a frontal lamp; and (4) sufficient energy controlling means which allow regulation of the required energy level and reading via a voltmeter The electronic component works with both direct and alternating current sufficient to meet the energy requirements of the apparatus of this invention. All the elements in the electronic component are contained inside a control box.

The optical and actinic light component comprises (1) a halogen light bulb (FIG. 1,2 010); (2) a collector lens with the halogen light bulb being positioned vertically and parallel to the collector lens (FIG. 1 020); (3) a means to position the light bulb which allows adjustment of the distance in two axes until the highest luminescent intensity is obtained in accordance with Kohler's luminescence characteristics described herein below.

The method of the present invention comprises the general steps of: (1) illumination, (2) excitement, and (3) suppression.

During the illumination step, the light emitted by the light bulb's filament (FIG. 1 010) passes through the collector lens (FIG. 1 020) which focuses the light beam which, in turn, is reflected at 90 degrees through a mirror placed for that purpose 45 degrees in the axis of the light's trajectory (FIG. 1,2 030, 030). It cannot be overemphasized that the apparatus and method of the present invention results in an episcopic illumination. Episcopic illumination is based on the illumination norms which adhere to optical and microscopic principles, including Kohler's illumination. Kohler's illumination allows the use of luminescent energy with minimal waste and heat emission.

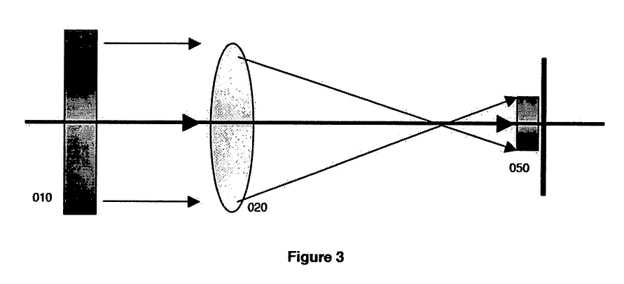

The light of this invention is by a luminescent source focused to the infinite (FIG. 1,3 010). It passes through a convergent or positive lens (FIG. 1, 3 020), which deviates the light beams to concentrate them in a focal plane (FIG. 3 050) resulting in an inverted image (FIG. 3 050) of a smaller diameter.

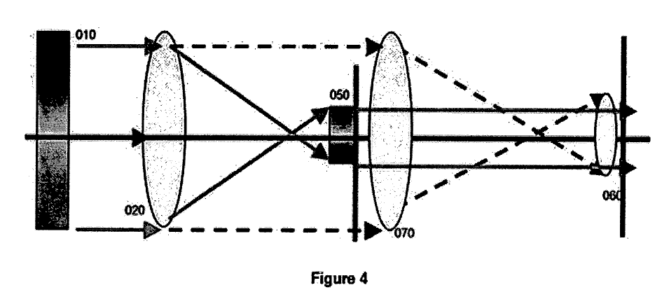

When two convergent collector lenses are used (FIG. 4), and the second collector lens, frontal of the objective (FIG. 1, 2, 3 070), concentrates the image projected by the first collector lens (FIG. 1,3,4 020), the image of the filament of the light source goes through without being seen or perceived at the focal concentrating plane, which will be the cervix (FIG. 4 060 ). In order to understand the method of the present invention one must take into account that the bulb's image comprises a spiral filament which passes through without being seen. Although the filament is invisible its energy can be used in an optimal manner because its beams in the second focal plane cross in a parallel manner contrary to the projected image (FIG. 4 050) of the first convergent collector lens (FIG. 4 020). The above described steps comprise Kohler's principle.

The first lens' image is projected on the focal plane's surface, the uterine cervix (FIG. 1,4 060). Smooth and colorless like crystal, the image looks like a uniform light field.

To fully understand the excitement state, one must first discern what “light” is. Our surroundings are full of waves. Humans can see or hear through waves, but our limited sensory capacity prevents us from detecting a vast array of waves. In the submicroscopic world, atoms and molecules are constituted of electrons, protons, mesotrons, photons, etc., which move in such a manner as to form waves in their bonds. Appropriately excited, those atoms and molecules emit waves that we call gamma rays, x-rays, ultraviolet waves, visible waves, television, radio, radiation and infrared, etc. (Electromagnetic Spectrum). The movements that are repeated by themselves on regular intervals are called periodic movements. Periodic movements include the sound of a pendulum clock, or the sound of a diapason. The vibration frequency is defined as the number of vibrations in one second. In other words, the frequency of vibration (v) and the period (T), reciprocate to one another: Frequency=1/Period and Period=1/Frequency. The same relationship expressed in algebraic symbols would be: v=1/T and T=1/v.

The movement of a body in graphic terms is often expressed in revolutions per minute (rpm), or revolutions per second (rps). Accordingly:

1 cycle/second=1 vibration/second=1 Hertz (Hz)

1 Hertz (Hz) is the unit.

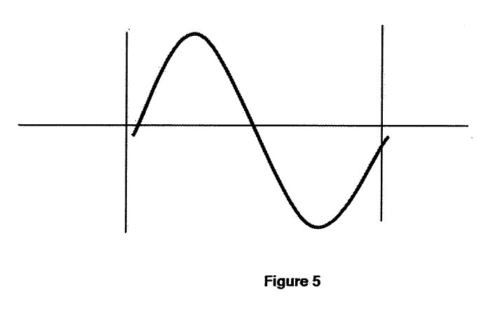

### Waves are described by their amplitude (crest to crest), frequency (Hz) and longitude (length) denominated λ (FIG. 5).

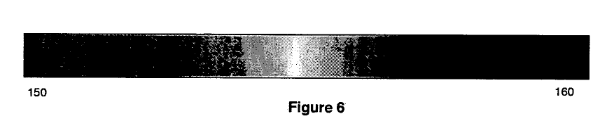

In the electromagnetic spectrum, light waves are designated according to their wave longitude, which is measured in nanometers (nm). One nanometer equals one billionth of a meter, or 10⁻⁹ meters. For example, the visible spectrum (FIG. 6 150 to 160 ranges from the deep red with a wave longitude of 700 nm to violet with a wave longitude of 400 nm.

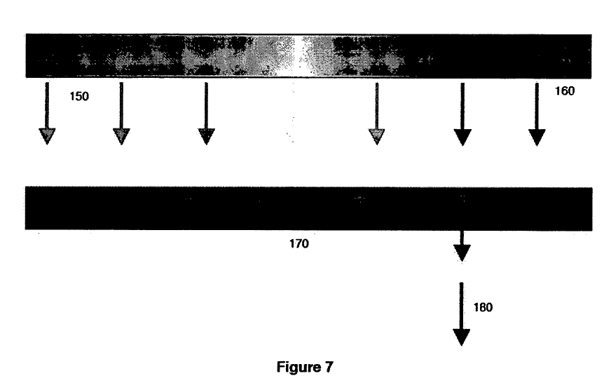

There are many types of light filters which allow different colors of the light spectrum to go through, based on their different wave longitude. There are green, blue, red, antethermic, etc. Most commercially available filters are not very discriminating and are unable to block all but one wave longitude. Specially designed and very costly filters are required to achieve such a high level of discrimination. The colposcopy of this invention requires a special narrow band pass interference filter (FIG. 7 040). That filter is known as the “exciter” filter and it is designed to pass light with a wave longitude of nanometers (FIG. 7 170).

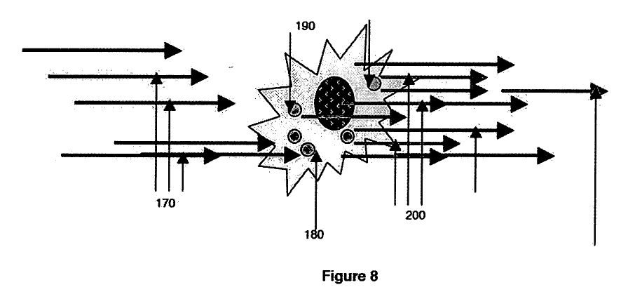

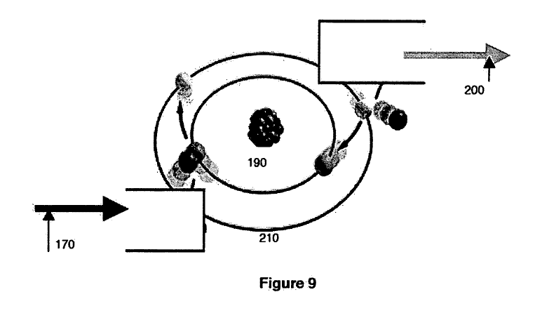

Below we describe the excitation of the fluorochrome in the colposcopy of this invention. First, the fluorochrome is applied using the method of this invention in the patient's cervix. Tissue cells infected with HPV absorb the fluorochrome while non-infected tissue cells do not absorb it (FIG. 8). A light beam of 488 nm wave longitude (FIG. 8 170) hits the fluorochrome inside the infected cells (FIG. 8 180, 190) and infected tissue. The flourochrome then absorbs in the infected tissues the light energy generated and emits light in different wave longitudes as compared with the light with which it was irradiated (FIG. 8 200). The physical phenomenon of fluorescence is produced: the tissue cells fluoresce. The energy of the light irradiated by the excitement filter at 488 nm (FIG. 7 040), is absorbed by atoms of fluorescein isothiocyanate (FIG. 9) inside the infected cells (FIG. 8,9 180, 190), exciting its electrons, changing its spin (FIG. 9 170, 210), and causing the emission of photons. A photon is produced when an electron's spin changes. In this case, photons with a wave longitude at 520 nm (FIG. 9 200) are produced. The photon's wave longitude is significantly different from the wave longitude with which the infected cells and tissue were irradiated originally. This effect is known as the Stokes-Adams Principle.

The following figure shows where, within the apparatus of this invention, the exciting filter (FIG. 1, 2 040) is located. It is an easy-to-set-up and easy-to-use system. The light beam goes through the exciting filter (FIG. 1, 2 040), and the ray goes through a first prism where it is sent to the frontal lens (FIG. 1, 2, 4 070) of the object, which condenses the light to then illuminate the point of the tissue being observed within the patient's lower genital tract (FIG. 1, 4 060).

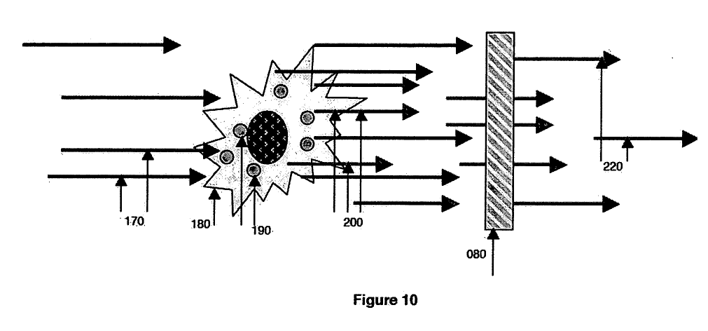

FIGS. 8 and 10 provide a specific example of epithelial cells organelles when they are excited. The luminescent energy irradiates the fluorochrome. That energy is then absorbed and converted in a light beam of a different wave longitude, as described before. The frontal lens then collects the energy and transmits it to the suppressing filters (FIG. 1, 2, 10 080), located inside the observational optic system of the colposcope. That system comprises a set of two objective lenses (FIG. 1 100), which send the image to the dull prisms (FIG. 1 120). The prisms then invert the image so that it can be analyzed through oculars (FIG. 1, 2 110, 120, 130). The colposcope of this invention also comprises a suppressor filter which sends the light beam to a video camera to be observed by the user through a monitor.

Suppressor filters are used to locate and see the fluorochrome in the infected cells and tissues. Specifically, a band pass filter allowing light with a wave longitude of 520 nm to pass through is suitable (FIG. 1, 2, 10 080). Two filters are required in the tri-dimensional stereoscopic system with oculars set up at 7 degrees of opening for each eye, and one filter is required for the video-photographic equipment. The reason for the filters is to allow passage of light of 520 nm frequency to be seen or recorded. This way the infected cells and tissues become unusually bright and can be distinguished from the healthy cells and tissues.

Having diagramed the epithelial cells being excited, the suppressor filter only allows for the light to go through at 520 nm. This way the zones where the fluorochrome is not captured will be dark, and, by contrast, the user will be able to clearly distinguish and see the zones where the flourochrome was captured, which coincide with the zones affected by the HPV-caused lesions also known as Dysplasia. Within the aforementioned context, a positive test is obtained when the HPV infected areas capture the fluorochrome, and a negative test result is obtained when there is no flourochrome.

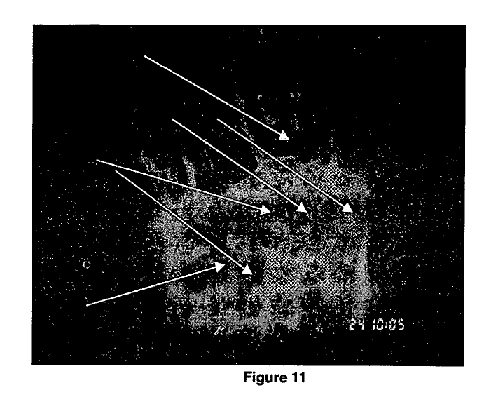

FIG. 11 illustrates a view of HPV induced lesions using the apparatus and method of the present invention. It is important to note that certain mucus tissue also shows up as fluorescent due to the cellular discarded materials, which should not be confused with the lesions indicated by the arrows. FIG. 11 involved a 50 year old patient which presented stage 3, positive biopsies. In that particular case, ordinary colposcopy failed to detect the HPV lesions.

### Preferred Embodiment of the Invention

In its preferred embodiment, the present invention uses the fluorochrome Fluorescein Isothiocyanate for several important reasons. This system is commonly used by ophthalmologists to conduct retinal fluorangiography, and in a solution to see corneal lesions. Ophthalmologists perform those procedures using an actinic lamp which has a cobalt filter and a wide spectral emission. The actinic lamp used by ophthalmologists does not have suppressor filters. The method of this invention uses a fluorochrome because, among other reasons, it does not present toxic or adverse effects in humans. In the clinical context, the method of this invention comprises the steps of:
setting the patient in the gynecological position;
inserting the plastic vaginal speculum to avoid undesirable reflexes;
visually locating the patient's cervix;
taking a vaginal pH swab (frequently HPV infections present with other bacterial illnesses or parasitical infections are characterized by alkaline pH):
taking samples of the endocervix with the citobrush;
taking samples of the ectocervix with the Ayre Spatula (speculum) or device;
uniformly preparing the sample trays to be sent to the laboratory to confirm the diagnosis of the method of this invention, if the ordering physician so requires; applying a 5% aqueous solution of acetic acid into the patient's cervix for one minute. The acidic solution has two functions: (1) bleaching the intraepithelial cervical neoplasia's cells; and (2) bonding the fluorochrome to the cells;
conducting a clear field colposcopy, which has approximately 70% accuracy and requires a rather long training time for prospective users;
introducing the green filter to see the vascular changes in the ordinary colposcope;
making the video-photographic logs to be able to compare images later;
uniformly applying the fluorescent isothiocyanate in the patient's cervix for one minute;
activating the colposcope's exciting filter.
activating the colposcope's suppressor filter, resulting in a “new” image which is obtained instantly;
making video-photographic logs of the “new” image using the camera mounted on the bolster; and
in case of positive results, and according to the degree and severity of the lesions disclosed by the “new” image, sending an affected area tissue sample in an Eppendore tube with the citobrush to a molecular biology laboratory for viral typing.

The method disclosed and claimed herein in the clinical context takes between 15 and 20 minutes.

What is claimed is:

1. A colposcope apparatus capable of detecting Human Papilloma Virus-produced lesions in the lower female genital tract and providing accurate and instant results, comprising:
a. a base further comprising a generally rectangular plate sufficiently large and heavy to provide support and stability to the apparatus, said plate comprising a top portion and a bottom portion, four independent rotating wells each engaged to the plate's bottom portion at each corner, said plate further comprising a brake mechanism which allows the apparatus to be moved and set in place;
b. a horizontal support comprising a first and a second end, the first end engaged to the top of the base as to allow rotating articulation of the horizontal support;
c. an optical arm engaged to the second end of the horizontal support comprising a counterbalanced mechanism which allows tri-axial movement permitting rotating articulation of the horizontal support and the optical arm, and to further allow maneuvering and setting the optical arm and any other element engaged to the optical arm in any precise position required by a gynecological procedure being performed;
d. a feeding source for illumination equipment held in place along the optical arm;
e. a bolster rotates and engages to the optical arm generally comprising a focusing system, said focusing system further comprising a calibrating mechanism which allows finely calibrated movement, an optical source of actinic light, said optical source further comprising an exciter filter, a prism, multiple suppressing filters, a halogen light bulb, said bulb further comprising a filament, a collector lens with the halogen light bulb being positioned vertically and parallel to the collector lens, means to position the light bulb which allows adjustment of the distance in two axes until the highest luminescent intensity is obtained in accordance with Kohler's luminescence characteristics, the bolster capable of transmitting and modulating luminescent energy which allows the user to see cervical images which can then be photographed;
f. a mirror placed 45 degrees in the axis of a light being emitted by the light bulb;
g. a power source capable of generating direct or alternate current that feeds a bulb located in the bolster;
h. two connectors of 120 V, alternate current suitable for a video camera and recorder;
i. a 12 volt, direct current contact suitable for use with a frontal lamp;
j. an energy controlling means which allows regulation of the required energy level and reading via a voltmeter;
k. a control box housing the power source, the connectors, the contact and the energy controlling means with sufficient controlling means to set the desired energy level;
l. an observational optic system comprising a set of two objective lenses capable of sending an image to multiple dull prisms, said prisms capable of inverting the image received so that it can be analyzed through oculars;
m. a suppressor filter capable of sending an image to a video camera to be observed by the user through a monitor.

2. A colposcope apparatus capable of detecting Human Papilloma Virus-produced lesions in the lower female genital tract, and providing accurate and instant results according to claim 1 wherein the bulb comprises a halogen photo optic lamp.

3. A colposcope apparatus capable of detecting Human Papilloma Virus-produced lesions in the lower female genital tract, and providing accurate and instant results according to claim 1 wherein the exciter filter comprises a narrow band pass interference filter.

4. A colposcope apparatus capable of detecting Human Papilloma Virus-produced lesions in the lower female genital tract, and providing accurate and instant results according to claim 1 wherein the collector lens is a convergent or positive lens.

5. A colposcope apparatus capable of detecting Human Papilloma Virus-produced lesions in the lower female genital tract, and providing accurate and instant results according to claim 1 wherein the bolster comprises two collector convergent lenses.

6. A method of using the colposcope apparatus of claim 1 to detect Human Papilloma Virus-produced lesions in the lower female genital tract and to provide accurate and instant results, said method comprising the steps of:
a. providing a colposcope apparatus as described and claimed in claim 1;
b. episcopicly illuminating the patient's area being studied according to the illumination norms which adhere to Kohler's optical and microscopic principles resulting in the use of luminescent energy with minimal waste and heat emission, said episcopic illuminating step further comprising passing a light beam emitted by the bulb's filament through the collector lens;
c. using the collector lens to focus the light beam;
d. reflecting the light beam 90 degrees through the mirror;
e. using a luminescent source to focus the light to the infinite;
f. passing the light through the lens, which deviates the light beams and concentrates them in a focal plane resulting in an inverted image of smaller diameter;
g. applying a fluorochrome solution to the patient's cervix to allow tissue cells infected with HPV to absorb the fluorochrome;
h. exposing the fluorochrome applied to the patient's cervix to a 488 nm wave longitude light beam thus allowing any cells infected with HPV to absorb the fluorochrome;
i. generating sufficient light energy to allow emission of light in different wave longitudes compared with the light used to irradiate the patient's cervix resulting in tissue cells fluorescing and production of fluorescein isothiocyanate inside the HPV-infected cells, said fluorescein isothiocyanate absorbing the energy of the light irradiated by the excitement filter at 488 nm inside the HPV-infected cells thus exciting the light's electrons, changing the light's spin, and causing the emission of photons with a wave longitude at 520 nm which is significantly different from the wave longitude with which the HPV-infected cells and tissue were irradiated originally, resulting in a physical effect known as the Stokes-Adams Principle;
j. collecting the energy being emitted using the frontal lens, said frontal lens transmitting the energy to the suppressing filters located inside the observational optic system of the colposcope;
k. using the suppressor filters to locate and see the fluorochrome in the patient's HPV-infected cells and tissues by virtue of the HPV-infected cells becoming unusually bright thus allowing the user to distinguish infected cells and tissue from healthy cells and tissue.

7. The method of using the colposcope apparatus of claim 1 to detect Human Papilloma Virus-produced lesions in the lower female genital tract and to provide accurate and instant results according to claim 6, wherein the band pass filter allows light with a wave longitude of 520 nm to pass through.

8. The method of using the colposcope apparatus of claim 1 to detect Human Papilloma Virus-produced lesions in the lower female genital tract and to provide accurate and instant results according to claim 6, wherein two suppressor filters are used to allow passage of light of 520 nm frequency resulting in a tri-dimensional stereoscopic system with oculars set up at 7 degrees of opening visible to the human eye.

9. The method of using the colposcope apparatus of claim 1 to detect Human Papilloma Virus-produced lesions in the lower female genital tract and to provide accurate and instant results according to claim 6, wherein one suppressor filter is used to allow passage of light of 520 nm frequency to be recorded by video-photographic equipment.

10. The method of using the colposcope apparatus of claim 1 to detect Human Papilloma Virus produced lesions in the lower female genital tract and to provide accurate and instant results according to claim 6, wherein two convergent collector lenses are used and the second collector lens, frontal of the objective concentrates an image projected by the first collector lens, an image produced by the filament of the light bulb going thru without being seen or perceived at a focal concentrating plane, said plane comprising a patient's cervix.
